(19) United States
(12) Patent Application Publication
(10) Pub. No.: US 2023/0077366 A1
(43) Pub. Date: Mar. 16, 2023

(54) EXTRUDED HONEYCOMB CATALYST

(71) Applicants: BASF SE, LUDWIGSHAFEN AM RHEIN (DE); BASF Corporation, Florham Park, NJ (US)

(72) Inventors: Feng ZHAO, SHANGHAI (CN); Sabine HUBER, Ludwigshafen (DE); Jia Di ZHANG, Shanghai (CN); Liang CHEN, Shanghai (CN); Diana Carolina GALEANO NUNEZ, Ludwigshafen (DE); Edgar Viktor HUENNEKES, Hannover (DE)

(21) Appl. No.: 18/049,347

(22) Filed: Oct. 25, 2022

Related U.S. Application Data
(63) Continuation of application No. 16/747,830, filed on Jan. 21, 2020.

(30) Foreign Application Priority Data
Dec. 30, 2016 (CN) PCT/CN2016/113637

(51) Int. Cl. A61B 18/14; A61B 18/12; A61B 17/34; A61F 7/00; A61B 17/16

(52) U.S. Cl. CPC A61B 18/1487; A61B 18/1206; A61B 17/3472; A61B 18/148; A61F 7/007; A61B 17/1642; A61B 17/1671; A61B 17/3403; A61B 17/3421; A61B 17/3468; A61B 18/1492; A61B 2018/126; A61B 2018/00577

(57) ABSTRACT

Disclosed are an extruded honeycomb catalyst, a process for preparing the catalyst, a method for reducing NOx in the exhaust gas from an internal combustion engine by using the catalyst, and a method for treatment of the emission gas generated from power plant comprising exposing the emission gas to the catalyst.

### TECHNICAL FIELD

Generally, the present invention relates to an extruded honeycomb catalyst, a process for preparing the catalyst, a method for reducing NOx in the exhaust gas from an internal combustion engine by using the catalyst, and a method for treatment of the emission gas generated from power plant comprising exposing the emission gas to the catalyst.

### BACKGROUND

NOₓ is one of the main exhaust gases of mobile source and stationary source which would be harmful to environment and human beings. In order to remove NOx from exhaust gases, catalytic reducing methods have heretofore been developed. The catalytic reducing methods are suitable for dealing with large quantities of exhaust gases, and of these, a process comprising adding ammonia as a reducing agent to catalytically reduce NOx selectively to N₂ is reported to be superior. The catalysts used in such a selective catalytic reduction (SCR) are required to reduce NOx over a broad temperature range like between 200° C. and 600° C. Moreover, SCR activity of these catalysts should not decrease dramatically after long-term hydrothermal and sulfur aging. V₂O₅/WO₃/TiO₂ catalysts have been well known in industry for its better S tolerance compared with Cu-Zeolite SCR. As mentioned in Applied Catalysis A: General, 80 (1992) page 135-148, WO₃ doping on V₂O₅/TiO₂ 1) increases the activity and widens the temperature window for SCR; 2) increases poison resistance to both alkali metal oxides and arsenious oxides; 3) reduces NH₃ oxidation as well as SO₂ oxidation.

With the enforcement of the more stringent NOx emission norms for the stationary and mobile applications in recent years, high performance and low cost NOx removal catalysts are extremely needed. Extruded honeycomb V₂O₅/WO₃/TiO₂ have been developed for the abatement of NOx as a high performance and low cost solution. Extruded honeycomb catalysts are one piece, monolithic objects which have a plurality of channels through which gas flows during the operation.

Previous publications U.S. Pat. No. 7,507,684B2, US 2014/0157763A1, WO 2010/099395 revealed the preparation of extruded honeycomb V₂O₅/WO₃/TiO₂ catalysts and their applications in NOx removal applications. Another publication WO 2013/179129 tried to claim extruded type wall flow catalysts consisting of (Aₓ)(T_{y})(R_{z})VO₄, where A is at least one alkaline earth metal, T is at least one transition metal, R is at least one rare earth metal, x, y, z are the molar ratios of each metal to vanadate (VO₄) with 1≥x, y, z≥0, x+y+z=1. However, there is not any example of the catalyst comprising V and Sb disclosed in WO 2013/179129.

WO 2013/017873A1 further discloses a coated extruded type of substrates or catalysts made with Fe-Beta zeolite, or V₂O₅/WO₃/TiO₂, or Fe-ZSM-5 (MFI) with another layer of Cu-SAPO, or SSZ-13, or WOₓ/CeO₂—ZrO₂ to further improve the functionality in different applications such as a SCR catalyst which is less sensitive to gas compositions.

SABIC filed a patent application US 2003/0144539A1 and claimed the structure of VSbₐM_{b}Oₓ and its applications in ammoxidation of alkanes and olefins, wherein M is at least one element selected from magnesium, aluminum, zirconium, silicon, hafnium, titanium and niobium, a is 0.5 to 20, b is 2 to 50, x is determined by the valence requirements of the elements present. Importantly, V and Sb were isolated in the matrix material M and did not form mixed oxides.

KR Pat. No. 101065242 and US Pat. No. 2009143225 disclose a SCR catalyst composition having improved NOx conversion at low temperature and the synthesis thereof, in which the catalyst has a formula of V₂O₅/Sb₂O₃/TiO₂, wherein the V/Sb binary system is supported on the support material. However, the formula and preparation method mentioned in US2009143225 could not produce the extruded honeycomb catalysts.

In U.S. Pat. No. 8,975,206B2, a supported XVO₄ structure (XVO₄/S) was disclosed, wherein X stands for Bi, Sb, Ga or Al etc., S is a support material comprising TiO₂, and only TiO₂/WO₃/SiO₂ was used as support in the examples.

Despite the work mentioned above, extruded honeycomb V-SCR catalysts using vanadium oxides as the active components and using antimony oxides or iron oxides as promoter have never been studied or disclosed.

### SUMMARY OF THE INVENTION

An object of the present invention is to provide a novel extruded honeycomb V-SCR catalyst. Compared with the traditional extruded honeycomb V₂O₅/WO₃/TiO₂ SCR catalyst, the newly designed catalyst showed better performance at broad temperature ranges and excellent thermal stability.

The object can be achieved by an extruded honeycomb catalyst, a process for preparing the catalyst, a method for reducing NOx in the exhaust gas from an internal is combustion engine by using the catalyst, and a method for treatment of the emission gas generated from power plant by using the catalyst.

In a first aspect of the invention, there provided an extruded honeycomb catalyst comprising vanadium oxides as the active component and antimony oxides or iron oxides as the promoter.

In a second aspect of the invention, there provided a process for preparing the catalyst of the present invention, comprising the steps of:
i) mixing vanadium oxides and/or the precursor thereof, antimony oxides and/or the precursor thereof, mixed antimony and vanadium oxides, mixed iron and vanadium oxides, the support and/or the precursor thereof, and the optional binder and/or matrix and/or the precursors thereof into a shapeable mixture;
ii) extruding the shapeable mixture into a flow-through honeycomb catalyst body;
iii) drying the catalyst body; and
iv) calcining the catalyst body.

In a third aspect of the invention, there provided a method for reducing NOx in the exhaust gas from an internal combustion engine, comprising contacting the exhaust gas with the catalyst of the present invention in the presence of a reductant, preferably NH₃.

In a fourth aspect of the present invention, there provided a method for treatment of the emission gas generated from power plant comprising exposing the emission gas to the catalyst.

Compared with traditional extruded honeycomb V₂O₅/WO₃/TiO₂ SCR catalyst, the inventive catalysts exhibit better performance broad temperature ranges and excellent thermal stability.

### BRIEF OF THE DRAWINGS

FIG. 1 shows the extruded honeycomb catalyst of the present invention.

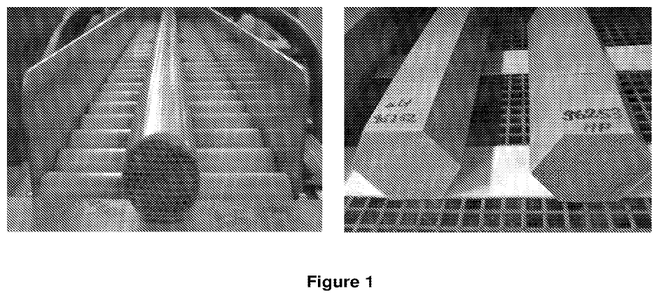

### DETAILED DESCRIPTION OF THE INVENTION

### <Extruded Honeycomb Catalyst>

In a first aspect of the invention, there provided an extruded honeycomb catalyst comprising vanadium oxides as the active component and antimony oxides or iron oxides as the promoter.

The vanadium oxides loading (calculated as V₂O₅) relative to the total weight of the catalyst ranges from 0.5 to 5 wt %, preferably from 1 to 5 wt %, more preferably from 1 to 3 wt %.

Sb in the catalyst is the promoter and used to improve the thermal stability of the active species vanadium oxides. The antimony oxides loading (calculated as Sb₂O₃) relative to the total weight of the catalyst ranges from 0.75 to 30 wt %, preferably from 1.5 to 15 wt %, more preferably 3 to 15 wt %.

V/Sb molar ratio can be from 8:1 to 1:8, more preferably from 6:1 to 1:3, and most preferably from 5:1 to 1:2.

The extruded catalyst of the present invention comprises active support materials. The active support materials for the active species vanadium oxides and the promoter antimony oxides include but not limited to: alumina, zirconia, titania, silica, silica alumina, silica titania, tungsten titania, silica tungsten titania, zeolite, ceria, ceria zirconia mixed oxides, and mixtures of any two or more above mentioned materials. Preferably, the support material comprises or more preferably consists of pure TiO₂, both of TiO₂ and SiO₂, or both of TiO₂ and WO₃, or TiO₂, SiO₂ and WO₃.

Furthermore, at least one binder and/or matrix components could be added to improve the mechanical strength of the final extruded products. The binder and/or matrix materials can be selected from group consisting of cordierite, nitrides, carbides, borides, intermetallic, aluminosilicate, spinel, alumina and/or doped alumina, silica, titania, zirconia, titania-zirconia, glass fiber and mixtures of any two or more thereof.

The active species in term of the total weight of vanadium oxides (calculated in the form of V₂O₅), antimony oxides (calculated in the form of Sb₂O₃), mixed antimony and vanadium oxides, mixed iron and vanadium oxides and the active support materials in percentages of total weight of the extruded catalysts can vary between 10 to 100%, preferably between 50 to 95%, more preferably between 70 to 90%, most preferably between 75 to 90%. The weight of additional binder and/or matrix materials content in the extruded catalyst can vary between 0 to 50%, preferably between 5 to 30%, most preferably from 10 to 25%, based on the total weight of the catalyst, so that the final products would combine the advantages of having good deNOx performance and enough mechanical strength at the same time.

The catalyst may further comprise other active components, such as at least one selected from antimony and vanadium mixed oxides such as SbVO₄, and iron and vanadium mixed oxides such as FeVO₄.

The catalyst of the present invention may take a form of a flow-through honeycomb catalyst body, i.e. with continuous flow channels. The flow channels of the honeycomb catalyst body are thin-walled channels, which can be of any suitable cross-sectional shape and size such as trapezoidal, rectangular, square, sinusoidal, hexagonal, oval, or circular. Such structures may contain up to 900 gas inlet openings (i.e., cells) per square inch (hereinafter abbreviated as cpsi) of cross section, wherein according to the present invention structures preferably have from 50 to 600 cpsi, more preferably from 200 to 600 cpsi, and even more preferably from 300 to 600 cpsi.

The inventive extruded honeycomb catalysts are one piece, monolithic objects which have a plurality of channels through which gas flows during operation. By the elimination of the ceramic substrate and the load of higher amount of catalytic active components compared with the coated catalysts, the extruded honeycomb catalysts have lower overall cost and will bring more active mass given the same catalyst volume, and thus render better performance at broad temperature ranges.

Another advantage is that, by using only one mass for extrusion, one eliminates the critical interphase between the ceramic substrate and the active coating. Even if the honeycomb is brittle to some amount, the active materials would not be lost.

### <Process for Preparing the Extruded Catalyst>

The second aspect of the present invention relates to a process for preparing the catalyst of the present invention.

The extruded catalyst may be prepared by a method including the steps of:
i) mixing the vanadium oxides and/or the precursor thereof, the antimony oxides and/or the precursor thereof, mixed antimony and vanadium oxides, mixed iron and vanadium oxides, the support and/or the precursor thereof, and the optional binder and/or matrix and/or the precursors thereof into a shapeable mixture;
ii) extruding the shapeable mixture into a flow-through honeycomb catalyst body;
iii) drying the catalyst body; and
iv) calcining the catalyst body.

In step i), at least one binder and/or matrix components could be added into the mixture to improve the mechanical strength of final extruded products. These materials could be selected from group consisting of cordierite, nitrides, carbides, borides, intermetallic, aluminosilicate, a spinel, alumina and/or doped alumina, silica, titania, zirconia, titania-zirconia, glass fiber and mixtures of any two or more thereof.

In step i) of the process, optionally any conventional additives could be added, such as plasticizer and/or dispersant etc. The suitable plasticizers are known for the person skilled in the art, such as polyethylene oxide or various kind of starch (such as WALOCEL from Dow Wolff Cellulosics GmbH, Germany, METHOCEL from Dow Wolff Cellulosics GmbH, Germany, cellulose ethers, carboxymethylcellulose, etc. or other functionalized carbohydrates (such as starch, dextrin, lactose, glucose, sugars or sugar alcohols being modified by ethoxylation or propoxylation, alkoxylated carbohydates, hydrogenated or partly hydrogenated carbohydrates and/or alkoxylated, hydrogenated or partly hydrogenated carbohydrates). The suitable dispersants are known for the person skilled in the art, such as graphite and comparable lubricants (such as polyethylene glycols, polyethylene oxide, methylcellulose, paraffin, stearic acid or stearate, carboxylic acid, silicone, petroleum oil, wax emulsions, lignosulfonates, etc.). The weight of the optional additives could be tuned for the extrusion operation, such as from 0.5 to 5%, preferably from 1 to 3%, based on the total weight of the catalyst.

In step i), optionally precipitator such as an organic acid could be added in order to peptize the powder mixture. The suitable organic acids are selected from the group consisting of formic acid, acetic acid or bifunctionalized acides such as oxalic acid, tartaric acid etc. The amount of the organic acids may be 1 to 20% by weight based on the total weight of the catalyst. The acids can be diluted or concentrated.

Moreover, in step i), optionally a pore forming agent could be added. The pore forming agent would decompose during the calcination of the catalyst and produce fine pores in the catalyst body. By selecting the type, the particle size and the amount of the pore forming agent, the number of the pores and the pore size could be controlled. The suitable pore forming agents are selected from the group of inorganic pore forming agents such as ammonium carbonate, ammonium bicarbonate, ammonium chloride salts, etc. or other thermally decomposable inorganic carbon such as graphite, coal ash, etc.) and/or pore organic forming agents consisting of carbohydrates with or without functional groups such as carboxy, hydroxyl such as fibers, polymers, polystyrene (PS), polymethyl methacrylate, etc.

The step i) may be carried out in the presence of a solvent. The solvent may be any suitable solvents known in the art, preferably a solvent comprising water, preferably the solvent being deionized water.

The step ii) may be carried out by means of any commercially available suitable extrusion devices.

The extrudate may take a form of a flow-through honeycomb catalyst body, i.e. with continuous flow channels. The flow channels of the honeycomb catalyst body are thin-walled channels, which can be of any suitable cross-sectional shape and size such as trapezoidal, rectangular, square, sinusoidal, hexagonal, oval, or circular. Such structures may have up to 900 cpsi, wherein according to the present invention structures preferably have from 50 to 600 cpsi, more preferably from 300 to 600 cpsi, and even more preferably from 350 to 600 cpsi.

After extrusion, the extrudate may be wrapped in foil and dried in air or freeze dried at −10 to −30° C. at a low pressure (such as from 0.3 to 10 mbar). The drying period could be from 1 hour to 6 months.

After drying, the resultant extrudate is calcined. The calcination temperature could be from 250 to 700° C., preferably 450 to 650° C. The calcination period could be from 10 minutes to 10 hours.

In the context of the invention, the precursor of the vanadium oxides and the precursor of the antimony oxides are intended to mean the compounds that can be converted by calcination under oxidizing conditions or otherwise to vanadium oxides and antimony oxides, respectively, subsequently in the process.

The precursor of the vanadium oxides may be selected from the group consisting of ammonium vanadate, vanadyl oxalate, vanadium pentoxide, vanadium monoethanolamine, vanadium chloride, vanadium trichloride oxide, vanadyl sulfate and vanadium antimonate.

The precursor of the antimony oxides may be selected from the group consisting of antimony acetate, ethylene glycol antimony, antimony sulfate, antimony nitrate, antimony chloride, antimony sulfide, antimony oxide and antimony vanadate.

### <Method for Reducing NOx in the Exhaust Gas>

The third aspect of the present invention relates to a method for reducing NOx in the exhaust gas from an internal combustion engine, comprising contacting the exhaust gas with the catalyst of the present invention in the presence of a reductant, preferably NH₃.

In an embodiment of the invention, the exhaust gas is contacted with the catalyst under a temperature in the range of 150 to 650° C., or 180 to 600° C., or 200 to 550° C.

The contact of the exhaust gas with the extruded catalyst is conducted in the presence of a reductant. The reductant that can be used in the present invention may be any reductants known in the art per se for reducing NOx, for example NH₃. NH₃ may be derived from urea.

There may be other catalyst upstream or downstream of the present invention, relative to the flow direction of the exhaust gas.

In a preferred embodiment of the invention, the internal combustion engine is a diesel engine.

### <Method for Reducing NOx in the Exhaust Gas>

The fourth aspect of the present invention relates to a method for treatment of the emission gas generated from power plant comprising exposing the emission gas to the catalyst.

The present invention is therefore directed to the following embodiments.
1. An extruded honeycomb catalyst, comprising:
a) vanadium oxides as the active component and antimony oxides as the promoter; or
b) mixed antimony and vanadium oxides; or
c) mixed iron and vanadium oxides.
2. The catalyst according to item 1, wherein it further comprises binder and/or matrix material.
3. The catalyst according to item 1 or 2, wherein the catalyst further comprises at least one active support selected from the group consisting of alumina, zirconia, titania, silica, silica alumina, silica titania, tungsten titania, silica tungsten titania, zeolite, ceria, ceria zirconia mixed oxides, and mixtures of any two or more above mentioned materials.
4. The catalyst according to item 3, wherein the active support is TiO₂ based material, preferably comprises or more preferably consists of TiO₂, mixture of TiO₂ and SiO₂, mixture of TiO₂ and WO₃, or mixture of TiO₂, SiO₂ and WO₃.
5. The catalyst according to any one of items 1 to 4, wherein based on the total weight of the catalyst, the vanadium oxides (calculated in the form of V₂O₅) are in an amount of 0.5 to 5%, preferably 1 to 5%, more preferably 1 to 3% by weight.
6. The catalyst according to any one of items 1 to 5, wherein based on the total weight of the catalyst, the antimony oxides (calculated in the form of Sb₂O₃) are in the amount of 0.75 to 30 wt %, preferably 1.5 to 15 wt %, more preferably 3 to 15
7. The catalyst according to any one of items 1 to 6, wherein the catalyst comprising vanadium oxides and antimony oxides and the Sb/V molar ratio is from 8:1 to 1:8, more preferably from 6:1 to 1:3, and most preferably from 5:1 to 1:2.
8. The catalyst according to any one of items 1 to 7, wherein the catalyst comprises up to 900 cells per square inch (below as cpsi) of cross section, preferably from 50 to 600 cpsi, more preferably from 200 to 600 cpsi, and even more preferably from 300 to 600 cpsi.
9. The catalyst according to any one of items 1 to 8, wherein based on the total weight of the catalyst, the total weight of vanadium oxides (calculated in the form of V₂O₅), antimony oxides (calculated in the form of Sb₂O₃), mixed antimony and vanadium oxides, mixed iron and vanadium oxides and the active support is in the range of 50 to 95%, preferably 70 to 90%, and more preferably 75 to 90%.
10. The catalyst according to any one of items 1 to 9, wherein the binder and/or matrix material is selected from at least one of cordierite, glass fiber, nitrides, carbides, borides, intermetallic, aluminosilicate, spinel, alumina and/or doped alumina, silica, titania, zirconia, titania-zirconia, and mixtures of any two or more thereof.
11. The catalyst according to any of items 1 to 10, wherein the weight ratio of the binder and/or matrix material is in the range of 0 to 50%, preferably between 5 to 30%, most preferably from 10 to 25%, based on the total weight of the catalyst.
12. The catalyst according to any of items 1 to 11, wherein the vanadium oxides (calculated in the form of V₂O₅) are in an amount of 1 to 5% by weight, the antimony oxides (calculated in the form of Sb₂O₃) are in the amount of 1.5-15% by weight, the total weight of vanadium oxides (calculated in the form of V₂O₅), antimony oxides (calculated in the form of Sb₂O₃), and the active support is in the range of 70 to 90%, the weight ratio of the binder and/or matrix material is in the range of 5 to 30%.
13. The catalyst according to any of items 1 to 11, wherein the vanadium oxides (calculated in the form of V₂O₅) are in an amount of 1 to 3% by weight, the antimony oxides (calculated in the form of Sb₂O₃) are in the amount of 3-15% by weight, the total weight of vanadium oxides (calculated in the form of V₂O₅), antimony oxides (calculated in the form of Sb₂O₃), and the active support is in the range of 75 to 90%, the weight ratio of the binder and/or matrix material is in the range of 10 to 25%.
14. A process for preparing the catalyst of any one of items 1 to 13, comprising the steps of:
   i) mixing the vanadium oxides and/or the precursor thereof, the antimony oxides and/or the precursor thereof, mixed antimony and vanadium oxides, mixed iron and vanadium oxides, the support and/or the precursor thereof, and the optional binder and/or matrix and/or the precursors thereof into a shapeable mixture;
   ii) extruding the shapeable mixture into a flow-through honeycomb catalyst body;
   iii) drying the catalyst body; and
   iv) calcining the catalyst body.
15. The process according to item 14, comprising the steps of:
   providing a solution or a mixture comprising vanadium oxides and/or the precursors thereof, antimony oxides and/or the precursors thereof, mixed antimony and vanadium oxides, mixed iron and vanadium oxides, the support and/or the precursors of, and the optional binder and/or matrix and the precursors thereof, and mixing the solution or the mixture to obtain a shapeable mixture;
   extruding the shapeable mixture into a flow-through honeycomb catalyst body with continuous channels and with a six-edge cross section exhibiting a cell density of 200 cpsi;
   wrapping the catalyst body in foil and drying it in air for 6 weeks or freeze drying at −10 to −30° C. at low pressure;
   calcining at a temperature of 600° C. for 1 to 6 hours to form a solid catalyst body.
16. The process according to item 14 or 15, wherein the precursor of the vanadium oxides is selected from the group consisting of ammonium vanadate, vanadyl oxalate, vanadium pentoxide, vanadium monoethanolamine, vanadium chloride, vanadium trichloride oxide, vanadyl sulfate and vanadium antimonate.
17. The process according to any one of items 14 to 16, wherein the precursor of the antimony oxides is selected from the group consisting of antimony acetate, ethylene glycol antimony, antimony sulfate, antimony nitrate, antimony chloride, antimonous sulfide, antimony oxide and antimony vanadate.
18. The process according to any one of items 14 to 17, wherein in the step i) a solvent comprising water is added and/or a pore forming agent is added.
19. The process according to any one of items 14 to 18, wherein in the step i) one or more conventional additives such as plasticizer and/or dispersant and/or precipitator is added.
20. A catalyst obtainable by the process of any one of items 14 to 19.
21. A method for reducing NOx in the exhaust gas from an internal combustion engine, comprising contacting the exhaust gas with the catalyst of any one of items 1 to 13 and 20 in the presence of a reductant, preferably NH₃.
22. The method according to item 21, wherein the exhaust gas is contacted with the catalyst under a temperature in the range of 150 to 650° C., 180 to 600° C., or 200 to 550° C.
23. The method according to item 21 or 22, wherein the internal combustion engine is a diesel engine.
24. A method for treatment of the emission gas generated from power plant, comprising exposing the emission gas to the catalyst of any one of items 1 to 13 and 20.

### EXAMPLES

The following examples are provided to illustrate the invention, but by no means are limitation to the invention.

The same oxidic starting materials and the same binder were used for the examples to investigate the performance of the different active components and compositions, of course there are various combinations of other starting materials for Sb- and/or V-compounds.

### <General Procedure for Preparing the Catalyst>

Mixed V/Sb oxides VSbO4 used in the Examples is prepared as following: 40.0 g V₂O₅ and 64.1 g Sb₂O₃ were mixed in 300 g DI water, and agitated to form a suspension. This suspension was spray dried at 200° C. to form a mixture of oxides.

Mixed V/Fe oxides VFeO₄ is from Treibacher.

Commercially available powdered antimony oxides (Sb₂O₃ from Campine), vanadium oxides (V₂O₅), VSbO₄ and VFeO₄ are mixed with TiO₂ based supports TiO₂ (DT51 from Crystal) or WO₃/TiO₂ (DT52 from Crystal) and Cordierite 808 M/27, as binder and/or matrix material and the plasticizers polyethylene oxide PEO Alkox E160 (2%) and Walocel MW15000 GB (1%) and processed with an aqueous solution of formic acid into a shapeable and flowable slip.

The shapeable mixture is extruded into a flow-through honeycomb catalyst body, i.e. with continuous channels and with a circular cross section exhibiting a cell density of 100 cpsi in an extrusion device from Handle. Subsequently, the catalyst body is wrapped in foil and dried in air for 6 weeks, then it was dried unwrapped until it showed no further weight loss.

Afterwards, the catalyst body is calcined at a temperature of 600° C. for 3 hours to form a solid catalyst body.

TABLE 1

| | | | | | Binder and/or | | |
|---|---|---|---|---|---|---|---|
| | TiO₂ | WO₃ | Sb₂O₃ | V₂O₅ | Matrix Material | 200° C. | 500° C. |
| Example | wt % | wt % | wt % | wt % | wt % | deNOx % | deNOx % |
| Example 1 | 80.00 | | 3.75 | 2.50 | 13.75 | 26.5 | 62.7 |
| Example 2 | 80.00 | | 6.50 (VSbO₄) | | 13.50 | 23.6 | 67.6 |
| Example 3 | 74.00 | 8.2 | 4.70 (VFeO₄) | | 13.10 | 11.9 | 73.4 |
| Example 4 | 72.50 | 8.0 | 6.50 (VSbO₄) | | 13.00 | 27.3 | 65.6 |
| Example 5 | 73.25 | | 11.25 | 2.50 | 13.00 | 33.0 | 63.9 |
| Example 6 | 75.75 | | 3.75 | 7.50 | 13.00 | 0.9 | −66.7 |
| Example 7 | 20.00 | | 3.75 | 2.50 | 73.75 | 5.8 | 7.2 |
| Example 8 | 60.00 | | 3.75 | 2.50 | 33.75 | 21.8 | 43.3 |
| Comparative | 75 | 9.0 | 0 | 2.5 | 13.50 | 27.0 | 38.5 |
| Example 1 | | | | | | | |

The obtained Catalysts was aged at 550° C. for 100 hours and evaluated on a reactor. All the catalysts were cut into 1 inch diameter and 3 inch long cores and placed in the fixed lab simulator for testing. During performance evaluation, catalytic activities of catalyst at both 200° C. and 500° C. were measured to understand the deNOx performance at both low and high temperatures. The feed gas was consisting of: 500 ppm NH₃, 500 ppm NO, 10% H₂O, 5% O₂ and balanced by N₂. The space velocity was 60,000 h⁻¹. Catalyst inlet temperature was first increased to 200° C. in feed gas. NH₃, NOx concentration at catalyst outlet was monitored & recorded until the concentration of both became stable. Then catalyst inlet temperature further ramped up to 500° C. and catalysts outlet NOx and NH₃ concentration were again monitored & recorded until they both became stable. In the evaluation, catalyst inlet NOx and NH₃ concentration were both 500 ppm and did not change. DeNOx % efficiency was calculated via below equation

deNOx%=100×(500 ppm−Outlet stable NOx)/500 ppm

The formulation of the catalyst in the Examples and Comparative Example and the respective deNOx performance at both low and high temperatures are listed in Table 1. The weight percentage of vanadium oxides is calculated in the form of V₂O₅. The weight percentage of antimony oxides is calculated in the form of Sb₂O₃.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1-24. (canceled)

25. A process for preparing a catalyst, the method comprising:
i) mixing the vanadium oxide and/or a precursor thereof, the antimony oxide and/or a precursor thereof, or the mixed antimony and vanadium oxide, or the mixed iron and vanadium oxide, optionally a support and/or a precursor thereof, and optionally a binder and/or matrix and/or a precursors thereof into a shapeable mixture;
ii) extruding the shapeable mixture into a flow-through honeycomb catalyst body;
iii) drying the catalyst body; and
iv) calcining the catalyst body.

26. The process according to claim 25, comprising:
providing a solution or a mixture comprising the vanadium oxide and/or the precursors thereof, the antimony oxide and/or the precursors thereof, or the mixed antimony and vanadium oxide, or the mixed iron and vanadium oxide, the optional support and/or the precursors of, and the optional binder and/or matrix and the precursors thereof, and mixing the solution or the mixture to obtain the shapeable mixture;
extruding the shapeable mixture into the flow-through honeycomb catalyst body with continuous channels and with a six-edge cross section exhibiting a cell density of 200 cells per square inch;
wrapping the catalyst body in foil and drying it in air for 6 weeks or freeze drying at a temperature of −10° C. to −30° C. at low pressure; and
calcining at a temperature of 600° C. for 1 to 6 hours to form a solid catalyst body.

27. The process according to claim 25, wherein the precursor of the vanadium oxide is used and the precursor of the vanadium oxide is chosen from ammonium vanadate, vanadyl oxalate, vanadium pentoxide, vanadium monoethanolamine, vanadium chloride, vanadium trichloride oxide, vanadyl sulfate and vanadium antimonate.

28. The process according to claim 25, wherein the precursor of the antimony oxide is used and the precursor of the antimony oxide is chosen from antimony acetate, ethylene glycol antimony, antimony sulfate, antimony nitrate, antimony chloride, antimonous sulfide, antimony oxide and antimony vanadate.

29. The process according to claim 25, wherein in the mixing i) a solvent comprising water is added and/or a pore forming agent is added.

30. The process according to claim 25, wherein in the mixing i) one or more conventional additives of plasticizer, dispersant, and precipitator are added.

31. A method for reducing NOx in an exhaust gas from an internal combustion engine, the method comprising contacting the exhaust gas with a catalyst prepared according to claim 25 in the presence of a reductant.

32. The method according to claim 31, wherein the exhaust gas is contacted with the catalyst under a temperature in the range of 150° C. to 650° C., 180° C. to 600° C., or 200° C. to 550° C.

33. The method according to claim 31, wherein the internal combustion engine is a diesel engine.

34. A method for treating an emission gas generated from power plant, the method comprising exposing the emission gas to a catalyst prepared according to claim 25.
